(19) United States
(12) Patent Application Publication
(10) Pub. No.: US 2005/0136452 A1
(43) Pub. Date: Jun. 23, 2005

(54) Methods for monitoring expression of polymorphic alleles

(75) Inventors: Stephen P.A. Fodor, Palo Alto, CA (US); Michael P. Mittmann, Palo Alto, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/959,009

(22) Filed: Oct. 4, 2004

Related U.S. Application Data
(60) Provisional application No. 60/508,392, filed on Oct. 3, 2003.

(51) Int. Cl. C12Q 1/68; C12P 19/34

(52) U.S. Cl. 435/6; 435/91.2

(57) ABSTRACT

Methods and arrays for monitoring and detecting allele specific expression of multiallelic loci are provided. The methods and arrays may be used for detecting allele specific expression patterns using hybridization to allele specific probes and sets of probes.

### FIELD OF THE INVENTION

The present invention provides arrays of probes that are capable of analyzing gene expression and genotype on the same array. The invention relates to diverse fields, including genetics, genomics, biology, population biology, medicine, and medical diagnostics.

### BACKGROUND

The past years have seen a dynamic change in the ability of science to comprehend vast amounts of data. Pioneering technologies such as nucleic acid arrays allow scientists to delve into the world of genetics in far greater detail than ever before. Exploration of genomic DNA has long been a dream of the scientific community. Held within the complex structures of genomic DNA lies the potential to identify, diagnose, or treat diseases like cancer, Alzheimer disease or alcoholism. Exploitation of genomic information from plants and animals may also provide answers to the world's food distribution problems.

Recent efforts in the scientific community, such as the publication of the draft sequence of the human genome in February 2001, have changed the dream of genome exploration into a reality. Genome-wide assays, however, must contend with the complexity of genomes; the human genome for example is estimated to have a complexity of 3×10⁹ base pairs. Novel methods of sample preparation and sample analysis that reduce complexity may provide for the fast and cost effective exploration of complex samples of nucleic acids, particularly genomic DNA.

Single nucleotide polymorphisms (SNPs) have emerged as the marker of choice for genome wide association studies and genetic linkage studies. Building SNP maps of the genome will provide the framework for new studies to identify the underlying genetic basis of complex diseases such as cancer, mental illness and diabetes. Due to the wide ranging applications of SNPs there is still a need for the development of robust, flexible, cost-effective technology platforms that allow for scoring genotypes in large numbers of samples.

All documents, i.e., publications and patent applications, cited in this disclosure, including the foregoing, are incorporated by reference herein in their entireties for all purposes to the same extent as if each of the individual documents were specifically and individually indicated to be so incorporated by reference herein in its entirety.

### SUMMARY OF THE INVENTION

Methods for detecting expression of a first and a second allele of a multiallelic genetic locus in a sample wherein the alleles comprise different alleles of a polymorphism are disclosed. The method comprises obtaining an RNA sample, amplifying the RNA to generate an amplification product; labeling the amplification product; hybridizing the labeled amplification product to an array that comprises probes that are complementary to the first allele and probes that are complementary to the second allele. The probes are allele specific so they are capable of distinguishing between the two alleles by hybridization. A hybridization pattern is obtained and used to determine if the first and second alleles are expressed in the sample. The array may further comprise probes that are complementary to non-polymorphic regions of a plurality of genes. In a preferred embodiment the array comprises probes that are complementary to non-polymorphic regions of multiallelic genetic loci. Preferably the array comprises both polymorphic and non-polymorphic probes that are complementary to each of a plurality of multiallelic genes.

In another embodiment kits for detecting expression of a first and second allele of a multiallelic genetic locus in a sample wherein the first and second alleles comprise different alleles of a first polymorphisms are disclosed. The kit may comprise an array that includes a plurality of probes that are perfectly complementary to a first allele or a multiallelic locus and probes that are perfectly complementary to a second allele of the multiallelic locus. The polymorphisms may be, for example, SNPs, insertions of 1 to 3 or more bases or deletions of 1 to 3 or more bases. The array may further comprise probe sets that are complementary to non-polymorphic sequences of the multiallelic locus. The array may interrogate more than 1000, 10,000, 100,000 or more than 1,000,000 polymorphisms.

In another embodiment a method for identifying a relationship between a first multiallelic gene and a second gene is disclosed. The genotype of one or more polymorphisms in the first gene is determined and the expression of the second gene is analyzed in the genetic background of the first gene.

### DETAILED DESCRIPTION OF THE INVENTION

### a) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication No. WO 99/36760) and PCT/US01/04285 (International Publication No. WO 01/58593), which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598 (U.S. Patent Application Publication 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NASBA). (See, U.S. Pat. Nos. 5,409,818, 5,554,517, and 6,063,603, each of which is incorporated herein by reference). Other amplification methods that may be used include: Qbeta Replicase, described in PCT Patent Application No. PCT/US87/00880, isothermal amplification methods such as SDA, described in Walker et al. 1992, Nucleic Acids Res. 20(7):1691-6, 1992, and rolling circle amplification, described in U.S. Pat. No. 5,648,245. Other amplification methods that may be used are described in, U.S. Pat. Nos. 5,242,794, 5,494,810, 4,988,617 and in U.S. Ser. No. 09/854,317, each of which is incorporated herein by reference. Other amplification methods that may be used are disclosed in U.S. Patent Application Publication No. 20030143599.

Additional methods of sample preparation and techniques for reducing the complexity of a nucleic sample are described in Dong et al., Genome Research 11, 1418 (2001), in U.S. Pat. No. 6,361,947, 6,391,592 and U.S. Ser. Nos. 09/916,135, 09/920,491 (U.S. patent Application Publication 20030096235), 09/910,292 (U.S. patent application Publication 20030082543), and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621, 10/063,559 (U.S. Publication No. 20020183936), 10/065,856, 10/065,868, 10/328,818, 10/328,872, 10/423,403, and 60/482,389.

### B. Definitions

The term “array” as used herein refers to an intentionally created collection of molecules which can be prepared either synthetically or biosynthetically. The molecules in the array can be identical or different from each other. The array can assume a variety of formats, for example, libraries of soluble molecules; libraries of compounds tethered to resin beads, silica chips, or other solid supports.

The term “array plate” as used herein refers to a body having a plurality of arrays in which each microarray is separated by a physical barrier resistant to the passage of liquids and forming an area or space, referred to as a well, capable of containing liquids in contact with the probe array.

The term “biomonomer” as used herein refers to a single unit of biopolymer, which can be linked with the same or other biomonomers to form a biopolymer (for example, a single amino acid or nucleotide with two linking groups one or both of which may have removable protecting groups) or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

The term “biopolymer” or sometimes refer by “biological polymer” as used herein is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above.

The term “biopolymer synthesis” as used herein is intended to encompass the synthetic production, both organic and inorganic, of a biopolymer. Related to a bioploymer is a “biomonomer”.

The term “cartridge” as used herein refers to a body forming an area or space referred to as a well wherein a microarray is contained and separated from the passage of liquids.

The term “combinatorial synthesis strategy” as used herein refers to a combinatorial synthesis strategy is an ordered strategy for parallel synthesis of diverse polymer sequences by sequential addition of reagents which may be represented by a reactant matrix and a switch matrix, the product of which is a product matrix. A reactant matrix is a l column by m row matrix of the building blocks to be added. The switch matrix is all or a subset of the binary numbers, preferably ordered, between l and m arranged in columns. A “binary strategy” is one in which at least two successive steps illuminate a portion, often half, of a region of interest on the substrate.

In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “effective amount” as used herein refers to an amount sufficient to induce a desired result.

The term “genome” as used herein is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “genotype” as used herein refers to the genetic information an individual carries at one or more positions in the genome. A genotype may refer to the information present at a single polymorphism, for example, a single SNP. For example, if a SNP is biallelic and can be either an A or a C then if an individual is homozygous for A at that position the genotype of the SNP is homozygous A or AA. Genotype may also refer to the information present at a plurality of polymorphic positions. The phenotype is the observable properties of an individual resulting from the individual's genotype. Phenotype may also be influenced by environmental factors.

The term haplotype refers to a particular pattern of SNPs or alleles that tend to be inherited together over time. Frequently the SNPs or alleles are found in a sequential organization on a single chromosome. Haplotyping involves grouping subjects by haplotypes, or particular patterns of SNPs, often sequential SNPs found on the same chromosome.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than about 1 M and a temperature of at least 25° C. For example, conditions of 5× SSPE (750 mM NaCl, 50 mM NaPhosphate, 5 mM EDTA, pH 7.4) and a temperature of 25-30° C. are suitable for allele-specific probe hybridizations or conditions of 100 mM MES, 1 M [Na⁺], 20 mM EDTA, 0.01% Tween-20 and a temperature of 30-50° C., preferably at about 45-50° C. Hybridizations may be performed in the presence of agents such as herring sperm DNA at about 0.1 mg/ml, acetylated BSA at about 0.5 mg/ml. As other factors may affect the stringency of hybridization, including base composition and length of the complementary strands, presence of organic solvents and extent of base mismatching, the combination of parameters is more important than the absolute measure of any one alone. Hybridization conditions suitable for microarrays are described in the Gene Expression Technical Manual, 2004 and the GeneChip Mapping Assay Manual, 2004.

The term “hybridization probes” as used herein are oligonucleotides capable of binding in a base-specific manner to a complementary strand of nucleic acid. Such probes include peptide nucleic acids, as described in Nielsen et al., Science 254, 1497-1500 (1991), LNAs, as described in Koshkin et al. Tetrahedron 54:3607-3630, 1998, and U.S. Pat. No. 6,268,490 and other nucleic acid analogs and nucleic acid mimetics.

The term “hybridizing specifically to” as used herein refers to the binding, duplexing, or hybridizing of a molecule only to a particular nucleotide sequence or sequences under stringent conditions when that sequence is present in a complex mixture (for example, total cellular) DNA or RNA.

The term “initiation biomonomer” or “initiator biomonomer” as used herein is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

The term “isolated nucleic acid” as used herein mean an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

The term “label” as used herein refers to a luminescent label, a light scattering label or a radioactive label. Fluorescent labels include, inter alia, the commercially available fluorescein phosphoramidites such as Fluoreprime (Pharmacia), Fluoredite (Millipore) and FAM (ABI). See U.S. Pat. No. 6,287,778.

The term “ligand” as used herein refers to a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (for example, opiates, steroids, etc.), hormone receptors, peptides, enzymes, enzyme substrates, substrate analogs, transition state analogs, cofactors, drugs, proteins, and antibodies.

The term “linkage disequilibrium” or sometimes refer by allelic association as used herein refers to the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles.

The term “microtiter plates” as used herein refers to arrays of discrete wells that come in standard formats (96, 384 and 1536 wells) which are used for examination of the physical, chemical or biological characteristics of a quantity of samples in parallel.

The term “mixed population” or sometimes refer by “complex population” as used herein refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

The term “monomer” as used herein refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

The term “mRNA” or sometimes refer by “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “nucleic acid library” or sometimes refer by “array” as used herein refers to an intentionally created collection of nucleic acids which can be prepared either synthetically or biosynthetically and screened for biological activity in a variety of different formats (for example, libraries of soluble molecules; and libraries of oligos tethered to resin beads, silica chips, or other solid supports). Additionally, the term “array” is meant to include those libraries of nucleic acids which can be prepared by spotting nucleic acids of essentially any length (for example, from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide.

Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term “polymorphism” as used herein refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. Insertions may be 1, 2 or 3 bases or more. Deletions may be 1, 2 or 3 bases or more. A polymorphic locus may be as small as one base pair. Polymorphic markers include restriction fragment length polymorphisms, variable number of tandem repeats (VNTR's), hypervariable regions, mini satellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms.

Single nucleotide polymorphisms (SNPs) are the most common source of genetic polymorphism in the human genome, accounting for approximately 90% of all human DNA polymorphisms. There are two types of substitutions resulting in SNPs: transitions where a purine is substituted for a purine (i.e. A for G) or a pyrimidine is substituted for a pyrimidine (i.e. C for T) and transversions where a purine is substituted for a pyrimidine or a pyrimidine for a purine. Transitions are more common than transversions. SNPs occur throughout the genome, including within the coding regions of genes and outside the coding regions in non-coding regions. The distribution of SNPs is not uniform, for example, there are fewer SNPs on average in the sex chromosomes than in the autosomal chromosomes and higher concentrations of SNPs are often found around specific locations within a chromosome. Within coding regions SNPs can be either synonymous or silent mutation, where the substitution causes no change to the protein or non-synonymous, where the mutation results in an alteration of the encoded amino acid. The alteration may be a missense mutation, resulting in a change to one or more amino acids in the protein, or a nonsense mutation, resulting in the introduction of a termination codon. Applications of SNPs include pharmacogenomics, diagnostic genomics, functional proteomics and therapeutic genomics.

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase.

The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

The term “reader” or “plate reader” as used herein refers to a device which is used to identify hybridization events on an array, such as the hybridization between a nucleic acid probe on the array and a fluorescently labeled target. Readers are known in the art and are commercially available through Affymetrix, Santa Clara Calif. and other companies. Generally, they involve the use of an excitation energy (such as a laser) to illuminate a fluorescently labeled target nucleic acid that has hybridized to the probe. Then, the reemitted radiation (at a different wavelength than the excitation energy) is detected using devices such as a CCD, PMT, photodiode, or similar devices to register the collected emissions. See U.S. Pat. No. 6,225,625.

The term “receptor” as used herein refers to a molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

The term “wafer” as used herein refers to a substrate having surface to which a plurality of arrays are bound. In a preferred embodiment, the arrays are synthesized on the surface of the substrate to create multiple arrays that are physically separate. In one preferred embodiment of a wafer, the arrays are physically separated by a distance of at least about 0.1, 0.25, 0.5, 1 or 1.5 millimeters. The arrays that are on the wafer may be identical, each one may be different, or there may be some combination thereof. Particularly preferred wafers are about 8″×8″ and are made using the photolithographic process.

### Method of Monitoring Differential Expression of SNPs

Predictions suggest that there are more than 3 million SNPs in the human genome, resulting in an average of 1 SNP every 1,000 bases. Many of these SNPs will fall within the coding regions of genes or within sequences that regulate the expression of a gene or the function of a protein. It is likely that many of the SNPs that contribute to phenotypes will be found within genes and many of these will be within the coding region of a protein where they may result in a change in an amino acid or the introduction of a new start or stop codon. Many genes will contain more than one polymorphism. It is likely that many of these polymorphisms will affect the expression of the genes that they are within, for example, by altering stability of the mRNA, splicing, capping, translation or polyadenylation. Methods of monitoring the impact of specific polymorphisms on the expression of genes are disclosed.

In diploid organisms, such as humans, different alleles of the same SNP may be present in an individual making that individual heterozygous for that SNP (A/B as opposed to homozygous A/A or B/B). The two different alleles may differentially affect the expression of the gene. Allele specific hybridization may be used to detect the presence of different polymorphic forms of an mRNA. The different polymorphic forms are highly homologous and may vary in as little as a single position. For example, if a gene has a single SNP in its mRNA that has two possible alleles, A and B where A is a T and B is a C, if the individual is heterozygous for the SNP they have one copy of allele A and one copy of allele B. The mRNA from allele A is identical to the mRNA from allele B except at the SNP there is a T in the mRNA from allele A and a C in mRNA from allele B. The polymorphism may have no effect on the expression of either alleles or it may result in differential expression or differential stability of one of the alleles. For example if the C in allele B results in aberrant splicing and destabilization of the mRNA from allele B then that allele may be underrepresented in the mRNA.

In one embodiment a method is provided for monitoring the expression of different alleles of a multiallelic locus by allele specific detection of transcribed RNA. For example, if a gene has two polymorphic forms varying at a SNP, having allele A or allele B. The genes may be differentially expressed so that if an organism is heterozygous at the SNP, (A/B) one of the alleles may be expressed at a higher level than the other. In one embodiment methods and arrays are provided for determining the effect that a particular SNP or SNP allele has on the expression of a particular gene. A SNP may affect the expression of the gene in which the SNP is located or a SNP may affect the expression of a distant gene.

Methods for genotyping and genotyping arrays are described in U.S. patent application Ser. Nos. 10/321,741, 10/442,021, 09/916,135, 09/961,709, 09/920,491, 60/483,050, filed Jun. 27, 2003 and 60/470,475 filed May 14, 2003, each of which is incorporated herein by reference. Genotyping methods are also disclosed in U.S. Pat. Nos. 6,361,947 and 6,586,186 which are incorporated by reference. U.S. patent application Ser. No. 10/321,741 discloses methods of detecting allelic imbalance. U.S. patent application Ser. No. 10/463,991 discloses methods of detecting regions of linkage disequilibrium using ancestral allele states. U.S. patent application Ser. No. 10/681,773 discloses high density genotyping arrays for analysis of human polymorphisms. U.S. patent application Ser. No. 10/272,155 discloses methods of genotyping using extension of locus specific amplification followed by generic amplification. U.S. patent application Ser. No. 10/913,928 discloses methods of copy number analysis using arrays. These methods may be uses in conjunction with the methods disclosed herein for analysis of copy number. Methods of synthesis of pools of oligonucleotides are disclosed in U.S. patent application Ser. No. 10/912,445. Methods of analysis of methylation status are disclosed in U.S. patent application Ser. No. 10/841,027. The methods disclosed herein may be used in conjunction with these methods for analysis of biological samples. Each of these patent applications is incorporated by reference herein in their entireties.

In one embodiment an array is provided with probe sets that are complementary to a plurality of genes. Some of the probe sets are complementary to genes in a polymorphic region so that if multiple alleles are present at the polymorphism a hybridization pattern may be obtained that may be analyzed to determine which alleles are being expressed and the relative level of expression of different alleles. In one embodiment an array comprises probes that are complementary to mRNAs that are known or predicted to be polymorphic. In one embodiment the array also comprises probes that are complementary to mRNAs in regions that are not polymorphic. If an mRNA is polymorphic these probes will not distinguish between different alleles and should detect mRNA from both alleles if present.

In a preferred embodiment the probes for distinguishing between RNA transcribed from different alleles of a multiallelic locus are tiled in a block of probes similar to the probes used in the Affymetrix GeneChip Mapping 10K Array, available from Affymetrix, Inc. See also, Mei et al. Genome Res. 2000 10: 1126-1137 and GeneChip Mapping Assay Manual, 2003, available from Affymetrix, Inc., Santa Clara, Calif., both of which are incorporated by reference. The probes are designed to distinguish between alleles of a polymorphism where the polymorphism is present in the RNA transcript. Probes are designed in blocks so that they are complementary to the mRNA in the region containing the SNP. In a preferred embodiment there are 40 probes per SNP, 20 for each allele. The probes may be organized in sets of 8 probes: perfect match (PM) and mismatch (MM) for each allele and for each strand. There are 5 sets of 8 probes, differing in the location of the polymorphic allele. In one set the polymorphic allele is at the central position (position 0) which in a 25 mer probe is the 13^{th} base. In the other sets the position of the polymorphism may be shifted to the 5′ or 3′ side of the central position, for example, the SNP may be the 9^{th} base in the 25 mer probe (position −4) or the 17^{th} base (position 4). The SNP position may be, for example, −4, −2, −1, 0, 1, 3 and 4. In a preferred embodiment the mismatch position is the central position, 0, in each of the probes. Other genotyping arrays are disclosed, for example, in U.S. patent application Ser. No. 60/585,352 filed Jul. 2, 2004. Methods for analysis of genotype are also disclosed in U.S. patent application Ser. Nos. 10/880,143 and 10/891,260.

In one embodiment expression of different alleles is analyzed by isolating RNA, amplifying the RNA by any method known to the art, labeling the amplification product and hybridizing the labeled amplification product to the array. Methods for amplification of RNA are well known in the art and are disclosed for example in U.S. patent application Ser. Nos. 10/821,024 and U.S. Pat. Nos. 5,514,545, 5,716,785, 6,582,938, 6,794,138 and 6,582,906.

In another embodiment probes to detect different alleles are tiled in multiple blocks of different length probes. Different lengths of probes hybridize with different stabilities. Shorter length probes are typically more sensitive to mismatch than longer probes so a probe that is 17 bases long may discriminate between two different alleles more effectively than a probe that is 25 bases long. In one embodiment probe sets of 17, 21 and 25 bases may be tiled. Probes between 17 and 30 bases may be tiled in another embodiment. Probes are designed to be specific for the individual alleles of the polymorphism to allow tuning of sensitivity. Shorter probes are less likely to cross-hybridized to the non-target allele but are also less likely to hybridize stably to the target allele. Longer probes bind more stably to their targets but are less sensitive to the presence of a mismatch and are more likely to cross hybridize to the non-target allele.

In another embodiment the probes are designed so that the polymorphic position is at different positions in the probe in different probes in the block, for example, the SNP may be at position 3, 6, 9, 12, 18 and 21. Discrimination between the alleles may vary with the location of the SNP position in the probe. The hybridization pattern is analyzed to determine which allele(s) of a polymorphism are being expressed.

In a preferred embodiment the array is designed to comprise probes to at least 1,000, more preferably 5,000, and more preferably 10,000 polymorphisms that are present in coding regions or expected to be present in RNA transcripts. The polymorphisms may be selected from the SNPs that are interrogated by the Affymetrix Mapping 10K or 100K arrays, see U.S. Provisional Application Nos. 60/417,190 filed Oct. 8, 2002 and 60/470,475 filed May 14, 2003 the disclosures of which are each incorporated herein by reference in their entireties. In another embodiment the SNPs are selected from the Affymetrix 100K SNP genotyping array SNPs, see U.S. Provisional Patent Application No. 60/585,352. The array further comprises probes to at least 5,000, 10,000, 20,000, 30,000 or 40,000 human genes. The array may, for example, comprise the probes that are present on the Affymetrix Human U133 array set, see U.S. patent application Ser. No. 10/355,577 which is incorporated herein by reference. The array thus has at least two types of probe sets, the first is capable of detecting expression of different alleles of multiallelic genes and the second set of probes detects transcription from a plurality of different genes but does not discriminate between different alleles. Probe sets that are capable of detecting alternative splicing events may also be included on the array. For example, probes that hybridize to a splicing junction may be included. The probes may recognize the junction between two exons after splicing or between an intron and an exon before splicing. Probes that recognize alternatively spliced junctions may also be included. In some embodiments probe sets that are specific for a single exon may be included on the array. A polymorphism may affect the splicing of an mRNA and in a preferred embodiment an array that simultaneously detects the genotype of a transcript and the spliced forms of that transcript are disclosed. For methods of detection of alternative splicing see, for example, U.S. patent application Ser. No. 60/536,315 filed Jan. 13, 2004.

In another embodiment a method of designing arrays with probe sets that discriminate between different alleles of a polymorphism and probe sets that do not discriminate is disclosed. The array may be used to simultaneously analyze the genotypes of transcripts to determine which alleles are present in the mRNA and to detect relationships between the genotype at one position and the expression of distant genes. The presence of a polymorphism in a first gene may impact the expression of a second gene or a group of genes that are different from the first gene. For example, if there is a polymorphism in a transcription factor that alters the function of the protein product this may impact the transcription of other genes. In one embodiment the disclosed array may be used to identify relationships between genes by identifying effects of a polymorphism on the expression of other genes.

Where the nucleic acid sample contains RNA, the RNA may be total RNA, poly(A)⁺ RNA, mRNA, rRNA, or tRNA, and may be isolated according to methods known in the art. (See, e.g., T. Maniatis et al., Molecular Cloning: A Laboratory Manual, 188-209 (Cold Spring Harbor Lab., Cold Spring Harbor, N.Y. 1982, which is expressly incorporated herein by reference.) The RNA may be heterogeneous, referring to any mixture of two or more distinct species of RNA. The species may be distinct based on any chemical or biological differences, including differences in base composition, length, or conformation. The RNA may contain full length mRNAs or mRNA fragments (i.e., less than full length) resulting from in vivo, in situ, or in vitro transcriptional events involving corresponding genes, gene fragments, or other DNA templates. In a preferred embodiment, the mRNA population of the present invention may contain single-stranded poly(A)⁺ RNA, which may be obtained from an RNA mixture (e.g., a whole cell RNA preparation), for example, by affinity chromatography purification through an oligo-dT cellulose column.

Methods of isolating total mRNA are well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993) and Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993), all of which are incorporated herein by reference in their entireties for all purposes.

GeneChip® nucleic acid probe arrays are manufactured using technology that combines photolithographic methods and combinatorial chemistry. In a preferred embodiment, over 1,000,000 different oligonucleotide probes are synthesized on each array. Each probe type is located in a specific area on the probe array called a probe cell or feature. Features may be, for example, (24 μm)², (18 μm)², (11 μm)², (6 μm)², (5 μm)² or smaller. Probe arrays may be packaged individually or in a multiple array format, for example, as part of a 96 array format.

Target Preparation-for a detailed protocol see GeneChip Expression Analysis Technical Manual (2003), available from Affymetrix, Inc., which is incorporated by reference. Also see Affymetrix Technical note, GeneChip Eukaryotic Small Sample Target Labeling Assay Version II, which is incorporated by reference.

In some embodiments the array is part of a kit to detect expression of different alleles of a multiallelic locus. The array may be designed to detect allele specific expression of a plurality of different SNP containing genes, for example, more than 1000, 10,000, 100,000 or 1,000,000 SNPs may be analyzed for expression differences. Computer executable code to determine from a hybridization pattern if a particular allele is being expressed in a particular sample may also be included in the kit.

In another embodiment the relationship between the genotype of one gene or allele specific expression of a gene and the expression of a second gene or group of genes is determined. In a preferred embodiment the first gene has a polymorphism in the coding region of the gene or in a regulatory region. Allele specific expression of the gene may result in expression effects in other genes and these may be detected by an array. The allele specific expression may be detected by hybridization to allele specific probe sets and simultaneously the effects on expression of other genes may be monitored by the non-allele specific probe sets. In one embodiment allele specific expression of a first gene may result in allele specific expression of a second gene or group of genes. This may be detected by the array containing a mixture of allele specific probes to SNPs in coding regions and the non-allele specific probes.

In some embodiments an mRNA may have two or more different polymorphisms. The array may comprise probes to detect one or more of the SNPs. In one embodiment detection of allele specific expression and genotype are determined simultaneously.

In some embodiments SNPs are selected for allele specific expression detection based on the method of amplification that will be used to amplify the RNA sample. Some methods of amplification amplify some sequences more efficiently than others and SNPs would be preferentially selected to be in regions that are efficiently amplified. Currently, the standard method of target preparation for GeneChip array analysis is to reverse transcribe RNA using an oligo(dT)-T7 promoter primer to synthesize first strand cDNA, synthesizing second strand cDNA with DNA polymerase and making multiple labeled copies of antisense RNA using T7 RNA polymerase. This method typically results in a bias toward the 3′ end of the starting mRNA. The bias is more prominent for longer mRNAs. As a result of the bias sequences in the 3′ end of the message are present at higher levels than sequences in the 5′ end of the mRNA so probes to the 3′ end of the mRNA are more likely to detect the antisense RNA. When using this method of amplification SNPs that are closer to the 3′ end of the mRNA are favored for targets over SNPs that are closer to the 5′ end of the mRNA.

Other methods of amplification may be unbiased, have reduced bias or be biased toward other regions of the mRNA. See, for example, U.S. patent application Ser. No. 10/090,320 and U.S. Pat. No. 6,251,639, which are incorporated by reference. Polymorphisms may be selected based on the amplification assay selected, for example, if using a 3′ biased amplification assay, polymorphisms that are within 600 bases of the 3′ end of the mRNA may be selected for analysis.

Genotypic analysis of SNPs provides important information about the association of genotype with phenotype. The disclosed methods provide methods of correlating genotype with changes in the expression of genes that are detectable at the RNA level. This may include, for example, changes in the rate of transcription, changes in splicing or processing events, or changes in the stability of the mRNA as a result of polymorphism. Such changes may be caused by non-coding or silent polymorphisms.

Pharmacogenomics applications may include, for example, correlating a patient's genotype with response to drug treatment. Treatments may be selected or optimized for a specific patient or population based on information about how a particular genotype responds to a particular drug treatment. Predictions of adverse drug response or inefficient drug therapy may be made based on genotype and correlated impact of genotype on expression. Drugs may be designed to correct or enhance the effects of mutations.

### CONCLUSION

Methods and arrays for detecting allele specific expression of polymorphic genes are disclosed. The arrays are particularly useful for determining which alleles of a particular polymorphism are expressed in a sample. The arrays and methods may also be used to study the effect of mutation on gene expression.

The above description is illustrative and not restrictive. Many variations of the invention will become apparent to those of skill in the art upon review of this disclosure. The scope of the invention should, therefore, be determined not with reference to the above description, but instead be determined with reference to the appended claims along with their full scope of equivalents.

1. A method of detecting expression of a first and a second allele of a multiallelic genetic locus in a sample wherein the first and second alleles comprise different alleles of a first SNP comprising:
obtaining an RNA sample, amplifying the RNA to generate an amplification product;
labeling the amplification product;
hybridizing the labeled amplification product to an array wherein the array comprises probes that are perfectly complementary to the first allele and probes that are perfectly complementary to the second allele;
obtaining a hybridization pattern; and,
determining from the hybridization pattern if the first and second alleles are expressed in the sample.

2. The method of claim 1 wherein the array further comprises a plurality of probes that are complementary to non-polymorphic regions of a plurality of genes.

3. The method of claim 2 wherein a subset of the plurality of genes is polymorphic.

4. A kit for detecting expression of a first and second allele of a multiallelic genetic locus in a sample wherein the first and second alleles comprise different alleles of a first polymorphism wherein the kit comprises: an array of probes comprising probes that are perfectly complementary to the first allele and probes that are perfectly complementary to the second allele.

5. An array of probes comprising a plurality of polymorphism probe sets wherein each polymorphism probe set comprises probes that are perfectly complementary to a first allele of a polymorphism and probes that are complementary to a second allele of the polymorphism and further comprising probe sets that are complementary to non-polymorphic sequences.

6. The array of claim 5 wherein there are at least 1000 polymorphism probe sets.

7. The array of claim 5 wherein there are at least 10,000 polymorphism probe sets.

8. The array of claim 5 wherein there are at least 100,000 polymorphism probe sets.

9. The array of claim 5 wherein there are at least 1,000,000 polymorphism probe sets.

10. The array of claim 5 wherein the polymorphism are selected from the SNPs present on the GeneChip 10K Mapping array and the array comprises probes from the Human Genome U133 expression array.

11. A method of identifying a relationship between two or more genes comprising: detecting the genotype of a first gene and determining the effect of said genotype on expression of a second gene.

12. A method of simultaneously detecting allele specific expression of a plurality of polymorphic genes and expression of a plurality of non-polymorphic genes comprising:
hybridizing a labeled nucleic acid sample to the array of claim 5;
detecting a hybridization pattern; and,
determining an expression pattern for a plurality of genes from said hybridization pattern.

13. A method of detecting the effect of allele specific expression of a first gene on the expression of a second gene comprising: obtaining an RNA sample; labeling said RNA; hybridizing the labeled RNA to an array of probes comprising a plurality of probe sets for genotyping a plurality of polymorphisms that are predicted to be in a transcribed region and probes that are complementary to a plurality of genes in non-polymorphic regions of the genes; and, identifying correlation between expression of a particular allele of a polymorphic gene and the expression level of at least one other gene.

14. The method of claim 12 wherein at least one of the polymorphisms is a non-SNP polymorphism.

15. The method of claim 14 wherein the non-SNP polymorphism is an insertion of 1, 2 or 3 bases.

16. The method of claim 14 wherein the non-SNP polymorphism is a deletion of 1, 2 or 3 bases.

17. The method of claim 14 wherein a subset of the plurality of genes is polymorphic.
